(19) United States
(12) Patent Application Publication
(10) Pub. No.: US 2006/0135983 A1
(43) Pub. Date: Jun. 22, 2006

(54) Catheter with tapered end balloon

(75) Inventors: Jeffry S. Melsheimer, Springville, IN (US); Thomas A. Osborne, Bloomington, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 11/313,593

(22) Filed: Dec. 13, 2005

Related U.S. Application Data
(60) Provisional application No. 60/636,819, filed on Dec. 16, 2004.

(51) Int. Cl. A61M 29/00

(52) U.S. Cl. 606/192; 623/1.11

(57) ABSTRACT

A delivery system, including a balloon catheter having a balloon with distal and proximal ends and a guide catheter for delivering the balloon catheter to a treatment site, is disclosed. The balloon tapers from at least one of the distal and proximal ends of the balloon to an active region on the surface of the balloon. The active region has a diameter D₁ which is greater than a diameter of the balloon at one or more of the distal and proximal ends of the balloon. Preferably, one or more of the lengths L₁, which is the distance from the proximal end of the balloon to the active region, and L₂, which is the distance from the distal end of the balloon to the active region, are between three to thirty times the diameter D₁.

This application claims priority under 35 U.S.C. § 119(e) to U.S. Provisional Patent Application No. 60/636,819, filed Dec. 16, 2004, which is hereby incorporated by reference herein.

### BACKGROUND

This invention relates generally to balloon catheters, and in particular, to a delivery system having a guide catheter which delivers a balloon catheter to a treatment site.

In the known delivery system, a balloon catheter having a balloon is delivered to a treatment site using a guide catheter. Such a balloon catheter is described in The American Journal of Cardiology, Vol. 49, Apr. 1, 1982, pages 1216 to 1222, and is employed to enlarge constrictions in vessels and body cavities, in particular coronary arteries. As is known in the art, balloon catheters may be used to deploy a stent at the constriction for the purpose of keeping the constriction open. At the tip of such a balloon catheter, an inflatable balloon is disposed, capable of being filled or emptied by way of a lumen inside the catheter.

Upon delivery to the treatment site, the balloon is deployed by withdrawing the guide catheter and then inflating the balloon. After inflating the balloon at the constriction, the balloon is deflated and retracted back into the guide catheter. Often, it may be difficult to deploy the uninflated balloon from the guide catheter, or to retract the deflated balloon back into the guide catheter after use. This difficulty may be attributed to various reasons such as the shape of the balloon, the balloon not completely deflating, or the balloon not returning to its initial folded configuration after deflation. As a result, the balloon may become caught against the guide catheter, making it difficult to either deploy the balloon catheter at the treatment site or remove the balloon catheter from the treatment site.

Therefore, a need exists for a delivery system having a balloon catheter which uses a balloon which is more easily deployed at the treatment site and retracted back into a guide catheter for removal from the treatment site.

### BRIEF SUMMARY

The present invention is defined by the following claims, and nothing in this section should be taken as a limitation on those claims. By way of introduction, the preferred embodiments described below relate to a balloon catheter. The balloon catheter includes a balloon having distal and proximal ends, an inflation lumen, and a guidewire lumen. The inflation lumen is formed in the balloon catheter and has distal and proximal ends and an opening at the distal end of the inflation lumen into an interior of the balloon. The inflation lumen is hermetically connected with the balloon at the proximal end of the balloon. The guidewire lumen is formed in the balloon catheter and is adapted to receive a guidewire in a slip-fit arrangement. The guidewire lumen traverses the interior of the balloon from the distal end to the proximal end of the balloon and is hermetically connected with the balloon at the distal end of the balloon. The balloon tapers from at least one of the distal and proximal ends of the balloon to an active region on the surface of the balloon. The active region has a diameter D₁ which is greater than a diameter of the balloon at one or both the distal and proximal ends of the balloon. Preferably, at least one of a length L₁, from the proximal end of the balloon to the active region, and a length L₂, from the distal end of the balloon to the active region, is between three to thirty times the diameter D₁.

The preferred embodiments further relate to a delivery system. The delivery system includes a balloon catheter having a balloon with distal and proximal ends and a guide catheter for delivering the balloon catheter to a treatment site. The balloon tapers from at least one of the distal and proximal ends of the balloon to an active region on the surface of the balloon. The active region has a diameter D₁ which is greater than a diameter of the balloon at one or more of the distal and proximal ends of the balloon. Preferably, at least one of a length L₁, from the proximal end of the balloon to the active region, and a length L₂, from the distal end of the balloon to the active region, is between three to thirty times the diameter of D₁.

The preferred embodiments further relate to a balloon catheter including a balloon having distal and proximal ends. The balloon tapers from at least one of the distal and proximal ends of the balloon to an active region on the surface of the balloon. The active region has a diameter D₁ which is greater than a diameter of the balloon at one or more of the distal and proximal ends of the balloon. Preferably, at least one of a length L₁, from the proximal end of the balloon to the active region, and a length L₂, from the distal end of the balloon to the active region, is between three to thirty times the diameter D₁.

### BRIEF DESCRIPTION OF THE DRAWINGS

FIG. 1 depicts a cross-sectional side view of a distal portion of a delivery system which comprises a guide catheter and a balloon catheter, in accordance with one preferred embodiment of this invention.

FIG. 2 depicts a cross-sectional view of the delivery system shown in FIG. 1 taken along line 2-2.

FIG. 3 depicts a partial cross-sectional view of the delivery system shown in FIG. 1 as it delivers a stent to a vessel.

FIG. 4 depicts a partial cross-sectional view of the delivery system shown in FIG. 1 with the balloon catheter deflated for retraction into the guide catheter.

It should be appreciated that for simplicity and clarity of illustration, elements shown in the Figures have not necessarily been drawn to scale. For example, the dimensions of some of the elements are exaggerated relative to each other for clarity. Further, where considered appropriate, reference numerals have been repeated among the Figures to indicate corresponding elements.

### DETAILED DESCRIPTION OF THE DRAWINGS AND THE PRESENTLY PREFERRED EMBODIMENTS

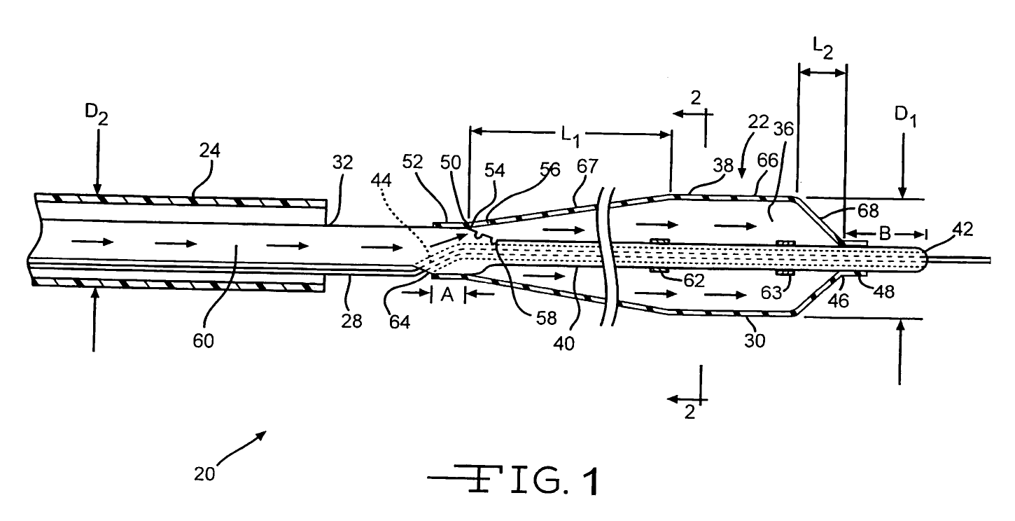

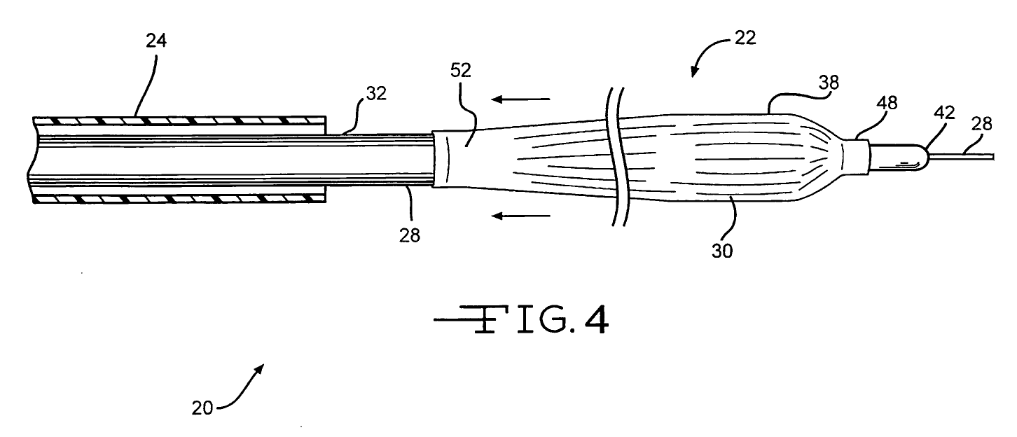

Referring to FIG. 1, there is shown a cross-sectional side view of a distal portion of a delivery system 20 which comprises a guide catheter 24 and a balloon catheter 22, according to one preferred embodiment. The delivery system 20 is designed to deliver the balloon catheter 22 to a treatment site, such as a vessel 70, with the aid of the guide catheter 24, as illustrated in FIGS. 1 and 3. The treatment site is any site to which a balloon catheter may be delivered, and includes vessels and body cavities, and in particular coronary arteries. Preferably, guide catheter 24 has a diameter D₂ of between 0.1 and 10 mm and a length of between about 100 to 1,500 mm.

Through the guide catheter 24, a guidewire 28 may be first advanced into the corresponding vessel 70. Preferably, the guidewire 28 is between 1,500 mm and 2,000 mm in length. The guidewire 28 serves as a pathway to guide the balloon catheter 22. The guidewire 28 may have a central lumen, not shown, for pressure measurement or to allow contrast injection.

The balloon catheter 22 includes a balloon 30 having a distal end 46 and a proximal end 50. As may be seen in FIG. 1, the balloon 30 is defined by an envelope 38, which is an outer surface of the balloon 30, and a length of guidewire lumen 40, wherein the guidewire lumen 40 forms a passage 44 sealed off from an interior 36 of the balloon 30. The passage 44 enables the balloon 30 to be thrust onto the guidewire 28 and thereby guided along the guidewire 28. To minimize frictional resistance between the interior of the passage 44 and the surface of the guidewire 28, the inside of the passage 44 and/or the outer surface of the guidewire 28 may be provided with a lubricant coating.

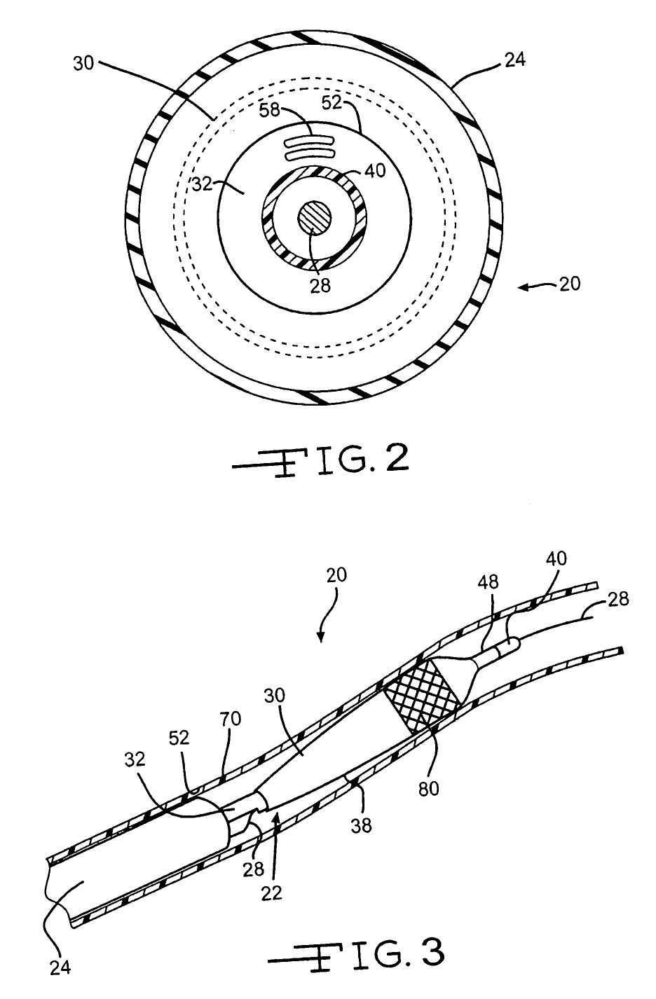

In FIG. 2, the substantially annular cross section of the balloon 30 is seen, together with the balloon passage 44 through which the guidewire 28 extends. For good transmission of the forces exerted upon the inflation lumen 32 to the balloon 30, a stabilizing wire, not shown, may extend into the neighborhood of a distal end 46 of the balloon 30.

As seen in FIG. 1, at the distal end 46 of the balloon 30, the envelope 38 takes the form of a length of flexible tubing 48, tightly sealed to the distal end of a segment of guidewire lumen 40. Similarly, the envelope 38 terminates at a proximal end 50 of the balloon in a length of flexible tubing 52, hermetically connected to a proximal end of the guidewire lumen 40 and to the inflation lumen 32.

Preferably, the balloon 30 is between 5 and 100 mm in length, and the inflation lumen 32 is between 100 and 1,500 mm in length. The balloon 30 may be made of Polyethylene, Polyethyleneterathylate (PET), Polyurethane, or any polymer or other suitable material known in the art. Preferably, the balloon 30 tapers from both the distal and the proximal ends 46, 50 to an active region 66 on the surface of the balloon 30, as illustrated in FIG. 1. The active region 66 is preferably cylindrical as shown. In other embodiments, the balloon 30 may be tapered at only one of the distal and proximal ends 46, 50. The active region 66 is the region of the balloon 30 which engages a vessel 70 or a stent 80 which is secured on the balloon 30, as illustrated in FIGS. 1 and 3. Preferably, the active region 66 has a diameter D₁ when the balloon is expanded, which is greater than a diameter of the balloon 30 at both the distal and proximal ends 46, 50 of the balloon 30. In other embodiments, the active region 66 may have a diameter D₁ which is greater than one of the diameters of the balloon 30 at the distal and proximal ends 46, 50 of the balloon 30. Preferably, the diameter D₁, in the active region 66, is greater than any other diameter of the balloon 30. Preferably, the diameter D₁ in the active region 66 is between 0.50 and 50 mm, and more preferably between 1 and 5 mm, and most preferably, between 1 and 3 mm. Preferably, the diameter D₂ of the guide catheter 24 is less than the diameter D₁ of the active region 66.

In a preferred embodiment, the taper from the distal end 46 to the active region 66 is referred to herein as a distal taper 68, and the taper from the proximal end 50 to the active region 66 is referred to herein as a proximal taper 67. The distal taper 68 and/or the proximal taper 67 may be straight, concave, or convex. The proximal taper 67 has a length L₁ from the proximal end 50 of the balloon 30 to the active region 66, while the distal taper 68 has a length L₂ from the distal end 46 of the balloon 30 to the active region 66. Preferably, the lengths L₁ and L₂ are measured in a direction generally parallel to the guidewire lumen 40, as illustrated in FIG. 1. However, in one embodiment, the lengths L₁ and L₂ are measured, respectively, in a direction along the envelope 38 from the proximal end 50 to the active region 66, and in a direction along the envelope 38 from the distal end 46 to the active region 66. In a preferred embodiment, one or more of the lengths L₁ and L₂ are between three to thirty times the diameter D₁ in the active region 66, and more preferably, between ten to thirty times the diameter D₁ in the active region 66, and most preferably, between ten to twenty times the diameter D₁ in the active region 66. As shown, it is preferable for the length L₁ of the proximal taper 67 to be longer than the length L₂ of the distal taper 68. For example, the length L₁ of the proximal taper 67 may be between three to thirty times the diameter D₁, and the length L₂ of the distal taper 68 may be about two times the diameter D₁ or less. The length L₁ may also be between about ten to thirty times the diameter D₁ or between about ten and twenty times the diameter D₁, while the length L₂ may also be about one times the diameter D₁ or less. Thus, the lengths L₁, L₂ do not need to be the same length, and the proximal and distal tapers 67, 68 may be asymmetrical with respect to each other. By having one or more of the proximal taper 67 and the distal taper 68 with the above dimensions, a user may more easily deploy and retract the balloon 30, and the balloon catheter 22, from and into the guide catheter 24. For instance, a distal taper 68 makes it easier for the user to deploy the balloon 30 from the guide catheter 24 prior to the balloon 30 being inflated, while a proximal taper 67 aids the user in retracting the balloon 30 back into the guide catheter 24 after the balloon 30 has been used and deflated, as discussed herein.

In one embodiment, the balloon catheter 22 includes an inflation lumen 32 having distal and proximal ends and a radial opening 58 adjacent the distal end of the inflation lumen 32 into an interior 36 of the balloon 30, as illustrated in FIGS. 1-4. FIG. 2 shows a section of the guidewire 28 and the inflation lumen 32. The balloon catheter 22 serves to transmit thrusts and tensions for pushing and pulling the balloon 30 to and from the desired location, and for rotating the balloon 30 around the guidewire 1. For this reason, it is desirable for the balloon catheter 22 to be reinforced by a stabilizing wire, not shown. The inflation lumen 32 is used to inject fluids 60, as shown by the direction of arrows in FIG. 1, into an interior 36 of the balloon 30 and for aspiration of fluids 60 when the diameter D₁ of the balloon 30 is to be decreased. The inflation lumen 32 supplies fluid 60 which is used to inflate the balloon 30. Preferably, the inflation lumen 32 is hermetically connected with the balloon 30 at the proximal end 50 of the balloon 30, so as to prevent fluid 60 from leaking out of the balloon 30.

An operative end 54 of balloon catheter 22, shown in FIG. 1, terminates in a taper 56. Preferably, both in the taper 56 and elsewhere at the operative end 54, a single, or a plurality of radial openings 58 are provided into the inflation lumen 32, whereby fluid 60 injected into the inflation lumen 32 can pass from the inflation lumen 32 into the interior 36 of the balloon 30.

In one embodiment, the balloon catheter 22 includes a guidewire lumen 40 adapted to receive a guidewire 28 in a slip-fit, coaxial arrangement. The guidewire lumen 40 traverses the interior 36 of the balloon 30 from the distal end 46 to the proximal end 50 of the balloon 30. Preferably, the guidewire lumen 40 is hermetically connected with the balloon 30 at the distal end 46 of the balloon 30, so as to prevent fluid 60 from leaking out of the balloon 30. In one embodiment, the guidewire lumen 40 forms a guidewire lumen opening 42 near the distal end 46 of the balloon 30 and a sideport opening 64 near the proximal end 50 of the balloon 30. The guidewire 28 enters the guidewire lumen 40 at the guidewire lumen opening 42 and exits the guidewire lumen 40 at the sideport opening 64.

In one embodiment, a distance A from the proximal end 50 of the balloon 30 to the sideport opening 64 is less than a distance B from the distal end 46 of the balloon 30 to the guidewire lumen opening 42. By having the distance A be less than the distance B, the balloon catheter 22 is more flexible at its central portion which allows the balloon catheter 22 to be more easily navigated through a vessel.

In one embodiment, gold striped bands 62 and 63 are located on the balloon catheter 22 around the guidewire lumen 40 serving to mark the location of the active region 66 in X-ray views, as shown in FIG. 1. In further embodiments, other radiopaque material such as tungsten or platinum may be used.

The delivery system 20 may deliver the balloon catheter 22 to any number of vessels, such as, for example, from a patient's right groin throughout the length of an artery to an aorta and to coronary arteries. The balloon catheter 22 may be used for a number of tasks, such as for example, delivering a stent to a constriction or stenosis in a vessel, or for dilation of coronary vessels. The balloon catheter 22 may be any type of balloon catheter which employs the use of a balloon, such as, but not limited to, over-the-wire systems, single-operator exchange systems, fixed wire systems, and perfusion balloons, as discussed in the Manual of Interventional Cardiology, Mark Freed et al., pgs. 22-24 (1996).

For dilatation of coronary vessels, the guidewire 28 is introduced through the guide catheter 24 into a vessel 70. The guidewire 28 lies freely in the guide catheter 24 and may be conveniently rotated and controlled. For anatomical orientation, additional doses of contrast medium may be supplied. When the guidewire 28 has passed a constriction or stenosis in the vessel 70, the tip of the guidewire 28 remains on the far side of a stenosis in the vessel 70. At this point, the balloon catheter 22 is thrust onto the guidewire 28 outside a patient's body and advanced through the guide catheter 24 along a track formed by the guidewire 28 into the vessel 70 and to the stenosis. If the balloon 30 is to be replaced during the procedure by a balloon 30 of larger size, it is a simple matter to retract the balloon catheter 22, leaving the distal end of the guidewire 28 positioned in the vessel 70 and permitting secure advancement of the replacement balloon to the location of the stenosis, without needing to overcome friction or reposition the guidewire 28. If insufficient stability as the result of dilatation is suspected, the guidewire 28 may even be left in place for an extended period of time, such as several hours, with the possibility to repeat dilatation at a later time. The distal end 46 of the balloon catheter 22 is flattened and wrapped in the manner described above for better insertability into vascular constrictions.

The invention permits the provision of balloons 30 of various lengths, widths and wall thicknesses to accommodate various pressures, and they may be interchanged with ease. Depending on medical requirements, the balloon catheters 22 are equipped with inflation lumens 32 of varying sizes. For larger balloon catheters 22, an additional inner lumen, not shown in the drawings, may be provided, its distal end extending to the distal end 46 of the balloon 30 and communicating with the interior of the vessel 70. In this way, pressure measurements and injections of contrast medium may be performed. The guidewire 28 may likewise be of different weights and flexibilities. The guidewire 28 may have soft, flexible tips, which may be shorter or longer, as well as straight or bowed. If no additional inner lumen is provided in the balloon catheter 22, a central lumen as above mentioned may be provided in the guidewires 28 for pressure measurements and injections of contrast medium. Once the balloon 30 is no longer needed, the balloon 30 is then deflated and retracted into the guide catheter 24, as illustrated in FIG. 4, and then pulled out of the vessel 70.

For delivering a stent 80 to a constriction or stenosis in a vessel, the balloon 30 and balloon catheter 22 are introduced into the vessel in much the same way as described above. However, for this procedure, the stent 80 is mounted around the active region 66 of the balloon 30, as illustrated in FIG. 3. Once the balloon 30 is delivered to the constriction or stenosis, the balloon 30 is inflated which causes the stent 80 to expand and be pressed against the inner wall of the vessel 70. The balloon is then deflated and retracted into the guide catheter 24, as illustrated in FIG. 4, leaving the expanded stent 80 at the site of the constriction or stenosis.

Although the invention has been described and illustrated with reference to specific illustrative embodiments thereof, it is not intended that the invention be limited to those illustrative embodiments. Those skilled in the art will recognize that variations and modifications can be made without departing from the spirit of the invention.

1. A balloon catheter comprising:
a balloon having distal and proximal ends;
an inflation lumen formed in the balloon catheter having distal and proximal ends and an opening at the distal end of the inflation lumen into an interior of the balloon, wherein the inflation lumen is hermetically connected with the balloon at the proximal end of the balloon; and
a guidewire lumen formed in the balloon catheter and adapted to receive a guidewire, the guidewire lumen traversing the interior of the balloon from the distal end to the proximal end of the balloon, wherein the guidewire lumen is hermetically connected with the balloon at the distal end of the balloon,
wherein the balloon tapers from at least one of the distal and proximal ends of the balloon to an active region on the surface of the balloon, the active region having a diameter D₁ which is greater than a diameter of the balloon at one or more of the distal and proximal ends of the balloon,
and wherein at least one of a length L₁, from the proximal end of the balloon to the active region, and a length L₂, from the distal end of the balloon to the active region, is between about three to thirty times the diameter D₁.

2. The balloon catheter of claim 1, wherein the balloon catheter forms both the inflation lumen and the guidewire lumen at the proximal end of the balloon.

3. The balloon catheter of claim 1, further comprising a stent surrounding the active region of the balloon, wherein the balloon is used to deliver the stent to a vessel.

4. The balloon catheter of claim 1, wherein the guidewire lumen forms a guidewire lumen opening near the distal end of the balloon and a sideport opening near the proximal end of the balloon.

5. The balloon catheter of claim 4, wherein a distance A from the proximal end of the balloon to the sideport opening is less than a distance B from the distal end of the balloon to the guidewire lumen opening.

6. The balloon catheter of claim 1, wherein at least one of the lengths L₁ and L₂ are between about ten to thirty times the diameter D₁.

7. The balloon catheter of claim 1, wherein the balloon tapers at both the distal and the proximal ends of the balloon to the active region on the surface of the balloon.

8. The balloon catheter of claim 7, wherein said length L₁ is between about three to thirty times the diameter D₁ and length L₂ is about two times the diameter or less.

9. The balloon catheter of claim 8, wherein said length L₁ is between about ten to twenty times the diameter D₁ and length L₂ is about one times the diameter or less.

10. A delivery system comprising:
a balloon catheter including a balloon having distal and proximal ends, wherein the balloon tapers from at least one of the distal and the proximal ends of the balloon to an active region on the surface of the balloon, the active region having a diameter D₁ which is greater than a diameter of the balloon at one or more of the distal and proximal ends of the balloon, and wherein at least one of a length L₁, from the proximal end of the balloon to the active region, and a length L₂, from the distal end of the balloon to the active region, is between about three to thirty times the diameter D₁; and
a guide catheter for delivering the balloon catheter to a treatment site.

11. The delivery system of claim 10, wherein the balloon catheter forms an inflation lumen having distal and proximal ends and an opening at the distal end of the inflation lumen into an interior of the balloon, wherein the inflation lumen is hermetically connected with the balloon at the proximal end of the balloon.

12. The delivery system of claim 10, further comprising a stent surrounding the active region of the balloon, wherein the balloon is used to deliver the stent to the treatment site.

13. The delivery system of claim 10, wherein the balloon catheter forms a guidewire lumen adapted to receive a guidewire in a slip-fit arrangement, the guidewire lumen traversing an interior of the balloon from the distal end to the proximal end of the balloon, wherein the guidewire lumen is formed in such a way as to remain sealed from communication with the balloon.

14. The delivery system of claim 13, wherein the guidewire lumen forms a guidewire lumen opening near the distal end of the balloon and a sideport opening near the proximal end of the balloon.

15. The delivery system of claim 14, wherein a distance A from the proximal end of the balloon to the sideport opening is less than a distance B from the distal end of the balloon to the guidewire lumen opening.

16. The delivery system of claim 10, wherein the guide catheter has a diameter D₂ that is less than the diameter D₁ of the active region.

17. The delivery system of claim 10, wherein the balloon tapers at both the distal and the proximal ends of the balloon to the active region on the surface of the balloon.

18. The balloon catheter of claim 17, wherein said length L₁ is between about three to thirty times the diameter D₁ and length L₂ is about two times the diameter or less.

19. The balloon catheter of claim 18, wherein said length L₁ is between about ten to twenty times the diameter D₁ and length L₂ is about one times the diameter or less.

20. A balloon catheter comprising:
a balloon having distal and proximal ends,
wherein the balloon tapers from at least one of the distal and the proximal ends of the balloon to an active region on the surface of the balloon, the active region having a diameter D₁ which is greater than a diameter of the balloon at one or more of the distal and proximal ends of the balloon,
and wherein at least one of a length L₁, from the proximal end of the balloon to the active region, and a length L₂, from the distal end of the balloon to the active region, is between about three to thirty times the diameter D₁.

21. The balloon catheter of claim 20, wherein the balloon catheter forms an inflation lumen having distal and proximal ends and an opening at the distal end of the inflation lumen into an interior of the balloon, wherein the inflation lumen is hermetically connected with the balloon at the proximal end of the balloon.

22. The balloon catheter of claim 20, further comprising a stent surrounding the active region of the balloon, wherein the balloon is used to deliver the stent to a treatment site.

23. The balloon catheter of claim 20, wherein the balloon catheter forms a guidewire lumen adapted to receive a guidewire in a slip-fit arrangement, the guidewire lumen traversing an interior of the balloon from the distal end to the proximal end of the balloon, wherein the guidewire lumen is connected in such a way as to remain sealed from communication with the distal and proximal ends of the balloon.

24. The balloon catheter of claim 23, wherein the guidewire lumen forms a guidewire lumen opening near the distal end of the balloon and a sideport opening near the proximal end of the balloon.

25. The balloon catheter of claim 24, wherein a distance A from the proximal end of the balloon to the sideport opening is less than a distance B from the distal end of the balloon to the guidewire lumen opening.

26. The balloon catheter of claim 20, wherein at least one of the lengths L₁ and L₂, are between about ten to thirty times the diameter D₁.

27. The balloon catheter of claim 20, wherein the balloon tapers at both the distal and the proximal ends of the balloon to the active region on the surface of the balloon.

28. The balloon catheter of claim 27, wherein said length L₁ is between about three to thirty times the diameter D₁ and length L₂ is about two times the diameter or less.

29. The balloon catheter of claim 28, wherein said length L₁ is between about ten to twenty times the diameter D₁ and length L₂ is about one times the diameter or less.
